(19) United States
(12) Patent Application Publication
(10) Pub. No.: US 2011/0009593 A1
(43) Pub. Date: Jan. 13, 2011

(54) Polymeric Bioplastics

(75) Inventors: Jon C. Clardy, Jamaica Plain, MA (US); Katherine N. Maloney, San Diego, CA (US); Frank C. Schroeder, Ithaca, NY (US)

Correspondence Address: FOLEY HOAG, LLP (w/HUV HMV);PATENT GROUP, 155 SEAPORT BLVD., BOSTON, MA, 02210-2600, US

(73) Assignee: President and Fellows of Harvard College, Cambridge, MA (US)

(21) Appl. No.: 12/681,448

(22) PCT Filed: Sep. 17, 2008
(86) PCT No.: PCT/US08/76622

§ 371 Date: Sep. 30, 2010

Related U.S. Application Data
(60) Provisional application No. 60/977,224, filed on Oct. 3, 2007.

(51) Int. Cl. C08G 63/00; C12P 7/62; C12N 1/14

(52) U.S. Cl. 528/271; 435/135; 435/254.1

(57) ABSTRACT

One aspect of the present invention relates to a polymeric material. The invention also relates to a method of making the aforementioned polymer comprising extracting a fungus, wherein the fungus is, for example, an endophytic fungus, such as CR873. The invention further relates to a bioplastic composition comprising the aforementioned polymer.

### RELATED APPLICATIONS

This application claims the benefit of priority to U.S. Provisional Patent Application Ser. No. 60/977,224, filed Oct. 3, 2007; the entirety of which is hereby incorporated by reference.

### GOVERNMENT SUPPORT

This invention was made with support provided by the NIH (NIH-NCDDG grant CA67786 and U19 CA 67786); therefore, the government has certain rights in the invention.

### BACKGROUND OF THE INVENTION

Bioplastics are a form of plastics derived from biological sources such as plant or bacterial, rather than traditional plastics, which are derived from petroleum. For example, in the medical area, degradable polymers have been developed that degrade in vivo into their respective monomers within weeks or a few months.

Polyhydroxyalkanoic acids (PHAs) are polyesters—made primarily by bacteria for carbon storage purposes—which have received great commercial interest as biodegradable, biocompatible polymers for a variety of uses.¹ Poly(3-hydroxybutyric acid) (poly(3HB)) is by far the most commonly found PHA. However, the homopolymer is unsuitable for most applications and PHAs incorporating new monomers with unique properties are desired.² At this time some 150 different hydroxyacid monomers have been observed in bacterial PHAs,³ the vast majority of which are esterified at a 3-hydroxy group. With the exception of poly(3HB) (small amounts of which are ubiquitous membrane components)⁴ and poly(malic acid),⁵ PHAs have been observed exclusively in prokaryotes.

While template-dependent polymer biosynthesis is a hallmark of all life, nontemplate-dependent polymer biosynthesis is an idiosyncratic phenomenon. The biosynthesis of polyhydroxybutyrate (poly(3-HB)), the polyester of 3-hydroxybutanoate (3-HB), by bacteria has become a paradigm for nontemplate-dependent polymer biosynthesis as shown in FIG. 3.1. Poly(3-HB) forms under nutrient-limited growth conditions with an ample carbon source. Under these conditions, glucose is converted to acetyl-CoA, which in turn is converted to 3-HB-CoA and poly(3-HB) by a series of relatively well-characterized enzyme steps. The poorly soluble poly(3-HB) forms granules that can constitute up to 80% of the dry cell weight.2 When conditions become more favorable, poly(3-HB) can be hydrolyzed to 3-HB, which enters the fatty acid P-oxidation pathway at the 3-hydroxy stage, to generate energy and reducing equivalents. By this strategy bacteria obtain (limited) energy from the carbon source and stockpile a material that can be utilized readily when nutrient limitations are lifted.

Poly(3-HB) and other polyhydroxyalkanoic acids (PHAs) have received much commercial interest as biodegradable, biocompatible polymers. 3-5 However the homopolymer of poly(3-HB) is unsuitable for most applications and PHAs incorporating new monomers with unique properties are desired. At this time some 150 different hydroxyacid monomers have been observed in bacterial PHAs, 7′ 8 the vast majority of which are esterified at a 3-hydroxy group. With the exception of membrane-associated poly(3-HB) (minute amounts of which are present in all organisms)' and poly(malic acid), lo, a PHAs have been observed exclusively in prokaryotes.

Catering products belong to the group of perishable plastics. Disposable crockery and cutlery, as well as pots and bowls, pack foils for hamburgers and straws are being dumped after a single use, together with food-leftovers, forming huge amounts of waste, particularly at big events. The use of bioplastics offers significant advantages not only in an ecological sense but also in an economical sense.

Constituting about 50 per cent of the bioplastics market, thermoplastic starch currently represents the most important and widely used bioplastic. Pure starch possesses the characteristic of being able to absorb humidity and is thus being used for the production of drug capsules in the pharmaceutical sector. Flexibiliser and plasticiser such as sorbitol and glycerine are added so that starch can also be processed thermo-plastically. By varying the amounts of these additives, the characteristic of the material can be tailored to specific needs (also called “thermo-plastical starch”).

Polylactide acid (PLA) is a transparent plastic made from natural resources. It not only resembles conventional petrochemical mass plastics (like PE or PP) in its characteristics, but it can also be processed easily on standard equipment that already exists for the production of conventional plastics. PLA and PLA-Blends generally come in the form of granulates with various properties and are used in the plastic processing industry for the production of foil, moulds, tins, cups, bottles and other packaging.

The biopolymer poly-3-hydroxybutyrate (PHB) is a polyester produced from renewable raw materials. Its characteristics are similar to those of the petrochemical-produced plastic polypropylene. Interest in PHB is currently very high. Companies worldwide are aiming to either begin production of PHB or to expand their current production capacity. Some estimate that this could result in a price reduction to fewer than 5 Euros per kilogram. Nevertheless, that is still four times the market price of polyethylene at February 2007. The South American sugar industry, for example, has decided to expand PHB production to an industrial scale. PHB is distinguished primarily by its physical characteristics. It produces transparent film at a melting point higher than 130 degrees Celsius, and is biodegradable without residue.

Poly HB, nevertheless, some disadvantages. It is not very soluble, so it is used as a thermoplastic (heated and injected into molds). Poly HB cannot be chemically modifed because it does not have reactive functional groups along the backbone of the polymer chain. It would be desirable to have a bioplastic which may be modified chemically in order to alter and tailor the properties of the polymer for particular uses.

PA 11 or Nylon 11 is a biopolymer derived from vegetable oil. It is also known under the tradename Rilsan®. PA 11 belongs to the technical polymers family and is not biodegradable. Its properties are similar than PA 12 although emissions of greenhouse gases and consumption of non-renewable resources are reduced during its production. Its thermal resistance is also superior than PA 12. It is used in high performance applications as automotive fuel lines, pneumatic airbrake tubing, electrical anti-termite cable sheathing, oil & gas flexible pipes & control fluid umbilicals, sports shoes, electronic device components, catheters, etc.

Polyhydroxyalkanoates are natural, thermoplastic polyesters and can be processed by traditional polymer techniques for use in an enormous variety of applications, including consumer packaging, disposable diaper linings and garbage bags, food and medical products. Initial efforts focused on molding applications, in particular for consumer packaging items such as bottles, cosmetic containers, pens, and golf tees. U.S. Pat. Nos. 4,826,493 and 4,880,592 describe the manufacture of poly-(R)-3-hydroxybutyrate (“PHB”) and poly-(R)-3-hydroxybutrate-co-(R)-3-hydroxyvalerate (“PHBV”) films and their use in diapers. U.S. Pat. No. 5,292,860 describes the manufacture of the PHA copolymer poly(3-hydroxybutyrate-co-3-hydroxyhexanoate) and the use of these polymers for making diaper backsheet film and other disposable items. Materials for manufacturing biodegradable personal hygiene articles or diapers from PHB copolymers other than PHBV are described in PCT WO 95/20614, WO 95/20621, WO 95/23250, WO 95/20615, WO 95/33874, WO 96/08535, and U.S. Pat. Nos. 5,502,116; 5,536,564; and 5,489,470.

Biodegradation of bioplastics, such as PHAs, is dependent upon a number of factors, such as the microbial activity of the environment and the surface area of the item. Temperature, pH, molecular weight, and crystallinity also are important factors. Biodegradation starts when microorganisms begin growing on the surface of the plastic and secrete enzymes which break down the polymer into hydroxy acid monomeric units, which are then taken up by the microorganisms and used as carbon sources for growth. In aerobic environments, the polymers are degraded to carbon dioxide and water, while in anaerobic environments the degradation products are carbon dioxide and methane. While the mechanism for degradation of PHAs in the environment is widely considered to be via enzymatic attack and can be relatively rapid, the mechanism of degradation in vivo is generally understood to involve simple hydrolytic attack on the polymers' ester linkages, which may or may not be protein mediated. Unlike polymers comprising 2-hydroxyacids such as polyglycolic acid and polylactic acid, polyhydroxyalkanoates normally are comprised of 3-hydroxyacids and, in certain cases, 4-, 5-, and 6-hydroxyacids. Ester linkages derived from these hydroxyacids are generally less susceptible to hydrolysis than ester linkages derived from 2-hydroxyacids. Researchers have developed processes for the production of a great variety of PHAs, and around 100 different monomers have been incorporated into polymers under controlled fermentation conditions. The commercially available PHAs, PHB and PHBV, represent only a small component of the property sets available in the PHAs.

Bioplastics such as PHB and PHBV have been investigated for use in medicine. These studies range from potential uses in drug delivery to use in formulation of tablets, surgical sutures wound dressings, lubricating powders, blood vessels, tissue scaffolds, surgical implants to join tubular body parts, bone fracture fixation plates, and other orthopedic uses. One advanced medical development is the use of PHB and PHBV for preparing a porous, bioresorbable flexible sheet for tissue separation and stimulation of tissue regeneration in injured soft tissue described in EP 7544467 A1 to Bowald et at. and EP 349505 A2.

Some implanted medical device should degrade after its primary function has been met. PHD and PHBV, the only PHAs tested as medical implants to date, have shown very long in vivo degradation periods, of greater than one year for PHB. For many applications, this very long degradation time is undesirable as the persistence of polymer at a wound healing site may lead to a chronic inflammatory response in the patient. Therefore, there is a need for medical implants with faster in vivo degradation rates.

Because of their biodegradability, the use of bioplastics is also especially popular in the packaging sector. The use of bioplastics for shopping bags is already very common. After their initial use they can be reused as bags for organic waste and then be composted. Trays and containers for fruit, vegetables, eggs and meat, bottles for soft drinks and dairy products and blister foils for fruit and vegetables are also already widely manufactured from bioplastics.

### SUMMARY OF THE INVENTION

The present invention is based, at least in part, on the discovery of novel polymeric compounds. These polymers, and methods of making and using them, are described herein. It is expected that these polymers will be useful as bioplastics. The bioplastics of the present invention are useful in various fields, including medical, surgical, pharmaceutical, cosmetics, foods and packaging.

The present invention relates to a novel polymer, a portion of which is represented by Formula 1:

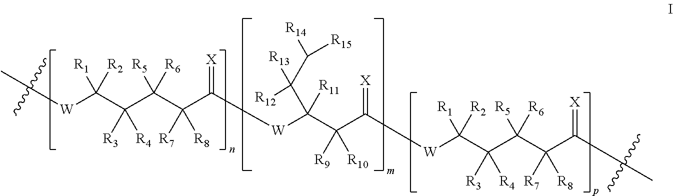

or a corresponding salt thereof, wherein:

X is independently for each occurrence O or S;

W is independently for each occurrence O, S, or NR₁₈;

R₁, R₅, and R₁₄ are each independently selected from the group consisting of OR₁₆, —O(O)R₁₆—CO(O)R₁₆, —C(O)NR₁₆R₁₇; —NR₁₆R₁₇, —NR(C)(O)R₁₆, sulfonamido, sulfoxy, sulfamyl, and halo;

R₂, R₃, R₄, R₆, R₇, R₈, R₉, R₁₀, R₁₁, R₁₂, R₁₃, and R₁₅ are each independently for each occurrence H, alkyl, alkyenyl, alkynyl, cycloalkyl, aryl, heterocyclyl, and heteroaryl;

R₁₆, R₁₇ and R₁₈ are each independently selected from the group consisting of alkyl, alkyenyl, alkynyl, cycloalkyl, aryl, heterocyclyl, and heteroaryl;

n, m and p are each independently an integer from 0 to 500, provided that n+m+p is equal to at least 2; and

wherein any alkyl, alkyenyl, alkynyl, cycloalkyl, aryl, heterocyclyl, or heteroaryl may optionally be substituted with hydroxy, alkoxy, alkenyloxy, alkynyloxy, aryloxy, aralkyloxy; halide, formyl, acetyl, halo, carboxyl, ester, cyano, nitro, SH, amino, amido, sulfonyl, or sulfonamido.

In another embodiment, the polymer represented by Formula II:

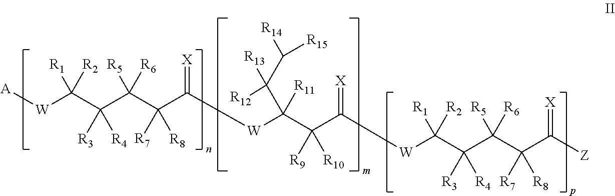

or a corresponding salt thereof,

wherein:

X is independently for each occurrence O or S;

W is independently for each occurrence O, S, or NR₁₈;

R₁, R₅, and R₁₄ are each independently selected from the group consisting of OR₁₆, —O(O)R₁₆—CO(O)R₁₆, —C(O)NR₁₆R₁₇; —NR₁₆R₁₇, —NR(C)(O)R₁₆, sulfonamido, sulfoxy, sulfamyl, nitro, cyano, and halo;

R₂, R₃, R₄, R₆, R₇, R₈, R₉, R₁₀, R₁₁, R₁₂, R₁₃, and R₁₅ are each independently for each occurrence H, alkyl, alkyenyl, alkynyl, cycloalkyl, aryl, heterocyclyl, and heteroaryl;

R₁₆, R₁₇ and R₁₈ are each independently selected from the group consisting of H, alkyl, alkyenyl, alkynyl, cycloalkyl, aryl, heterocyclyl, and heteroaryl;

n, m and p are each independently an integer from 0 to 500, provided that n+m+p is equal to at least 2; and

A and Z are independently for each occurrence selected from the group consisting of OR₁₆, —O(O)R₁₆—CO(O)R₁₆, —C(O)NR₁₆R₁₇; —NR₁₆R₁₇, —NR(C)(O)R₁₆, sulfonamido, sulfoxy, sulfamyl, nitro, cyano, halo; H, alkyl, alkyenyl, alkynyl, cycloalkyl, aryl, heterocyclyl, and heteroaryl; and

wherein any alkyl, alkyenyl, alkynyl, cycloalkyl, aryl, heterocyclyl, or heteroaryl may optionally be substituted with hydroxy, alkoxy, alkenyloxy, alkynyloxy, aryloxy, aralkyloxy; halide, formyl, acetyl, halo, carboxyl, ester, cyano, nitro, SH, amino, amido, sulfonyl, or sulfonamido.

In some embodiment, the polymers are represented by formulas I and II, wherein X and W are each O.

In some embodiments, R₁, R₅, and R₁₄ are for each occurrence OR₁₆.

In some embodiments, R₁, R₅, and R₁₄ are for each occurrence OH.

In some embodiments, R₂, R₃, R₄, R₆, R₇, R₈, R₉, R₁₀, R₁₁, R₁₂, and R₁₃ are each for occurrence H.

In some embodiments, n+m+p is equal to 5 to 250.

In some embodiments, n+m+p is equal to 5 to 100.

In some embodiments, n+m+p is equal to 5 to 50.

In some embodiments, n+m+p is equal to 5 to 25.

In some embodiments, m is equal to at least 1.

The polymers of the present invention are biodegradable.

In some embodiments, the polymer is soluble is a suitable solvent, such as alcohols (e.g. methanol or ethanol).

In other embodiments, the polymer is a thermoplastic, and may processed to form an article of manufacture using injection molding techniques.

In some embodiments, the polymer of the present invention has a molecular weight of about 200-100,000, 200 to 50,000, 500 to 25,000, 500 to 10,000 or 700 to 3000.

In some embodiments, R₁, R₅, and R₁₄ are independently for each occurrence selected from the group consisting of O(O)R₁₆—CO(O)R₁₆, —NR₁₆R₁₇, —NR(C)(O)R₁₆, sulfonamido, sulfoxy, and sulfamyl.

In some embodiments, A is H or alkyl, and Z are OR₁₆.

In another aspect of the invention, the polymer is represented by Formula IV:

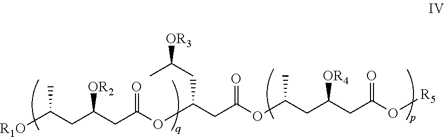

or a corresponding salt thereof,

wherein:

R₁, R₂, R₃, R₄, and R₅ are independently for each occurrence selected from the group consisting of H, alkyl, alkyenyl, alkynyl, cycloalkyl, aryl, heterocyclyl, and heteroaryl;

wherein any alkyl, alkyenyl, alkynyl, cycloalkyl, aryl, heterocyclyl, or heteroaryl may optionally be substituted with hydroxy, alkoxy, alkenyloxy, alkynyloxy, aryloxy, aralkyloxy; halide, formyl, acetyl, halo, carboxyl, ester, cyano, nitro, SH, amino, amido, sulfonyl, or sulfonamido; and

wherein p and q are each independently an integer from 1 to 500.

In some embodiments, p is equal to 1, and q is an integer from 1 to 500.

In some embodiments, q is an integer from 5 to 25.

In some embodiments, R₁, R₂, R₃, R₄, and R₅ are H.

In another aspect of the invention, the polymer comprises polymerized monomers represented by formula V:

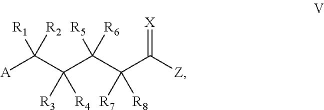

or a corresponding salt thereof,

wherein:

X is independently for each occurrence O or S;

W is independently for each occurrence O, S, or NR₁₈;

R₁, R₅, and R₁₄ are each independently selected from the group consisting of OR₁₆, —O(O)R₁₆—CO(O)R₁₆, —C(O)NR₁₆R₁₇; —NR₁₆R₁₇, —NR(C)(O)R₁₆, sulfonamido, sulfoxy, sulfamyl, nitro, cyano, and halo;

R₂, R₃, R₄, R₆, R₇, R₈, R₉, R₁₀, R₁₁, R₁₂, R₁₃, and R₁₅ are each independently for each occurrence H, alkyl, alkyenyl, alkynyl, cycloalkyl, aryl, heterocyclyl, and heteroaryl;

R₁₆, R₁₇ and R₁₈ are each independently selected from the group consisting of H, alkyl, alkyenyl, alkynyl, cycloalkyl, aryl, heterocyclyl, and heteroaryl;

n, m and p are each independently an integer from 0 to 500, provided that n+m+p is equal to at least 2; and

A and Z are independently for each occurrence selected from the group consisting of OR₁₆, SR₁₆, —O(O)R₁₆; —NHR₁₆, —NR(C)(O)R₁₆, and halo; and

wherein any alkyl, alkyenyl, alkynyl, cycloalkyl, aryl, heterocyclyl, or heteroaryl may optionally be substituted with hydroxy, alkoxy, alkenyloxy, alkynyloxy, aryloxy, aralkyloxy; halide, formyl, acetyl, halo, carboxyl, ester, cyano, nitro, SH, amino, amido, sulfonyl, or sulfonamido.

In some embodiments, the monomers are attached via an ester, thioester, or amide linkage.

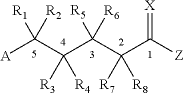

In some embodiments, at least one monomer is attached via an ester, thioester, or amide linkage at the 3 position, and the remaining monomers are connected at the 5 position, as shown above.

In some embodiments, the polymer has a molecular weight of about 200 to 200,000, 200 to 50,000, 200 to 25,000, 200 to 10,000, or 200 to 5,000.

In some embodiments, the monomer represented by formula V is copolymerized with at least one additional monomer selected from the group consisting of hydroxy acids, amino acids, aminoalcohols, sugars, diols, and triols, tetraols.

In some embodiments, the additional monomer is selected from the group consisting of glycolic acid, lactic acid, 2-hydroxyethoxy acetic acid, polyetheylene glycol and hyarulonic acid.

Another aspect if the invention is directed to a method of making a polymer comprising extracting a fungus to produce the polymer.

In some embodiments, the fungus is an endophytic fungus.

In some embodiments, the fungus is CR873.

In some embodiments, the method further comprises culturing the fungus prior to extraction.

In some embodiments, the extraction is a solid phase extraction.

In some embodiments, the polymer comprises at least one hydroxy group, and the method further comprises chemically modifying the hydroxy group.

In some embodiments, the hydroxy group is modified to a group selected from OR₁₆, —O(O)R₁₆—CO(O)R₁₆, —C(O)NR₁₆R₁₇; —NR₁₆R₁₇, —NR(C)(O)R₁₆, sulfonamido, sulfoxy, sulfamyl, nitro, cyano, and halo,

wherein R₁₆, and R₁₇ are each independently selected from the group consisting of H, alkyl, alkyenyl, alkynyl, cycloalkyl, aryl, heterocyclyl, and heteroaryl; and

wherein any alkyl, alkyenyl, alkynyl, cycloalkyl, aryl, heterocyclyl, or heteroaryl may optionally be substituted with hydroxy, alkoxy, alkenyloxy, alkynyloxy, aryloxy, aralkyloxy; halide, formyl, acetyl, halo, carboxyl, ester, cyano, nitro, SH, amino, amido, sulfonyl, or sulfonamido.

Another aspect of the invention relates to a polymer, which is the product produced by any of the aforementioned processes.

Another aspect of the invention relates to a bioplastic composition comprising a polymer of the present invention.

In some embodiments, the bioplastic composition further comprises at least one additive.

In some embodiments, the additive increases the biodegradability of the polymer.

In some embodiments, the additive decreases the biodegradability of the polymer.

In some embodiments, the additive is selected from the group consisting of inorganic acids, inorganic bases, ammonium salts, organic acids, and surfactants.

In some embodiments, the additive is selected from the group consisting ammonium sulfate, ammonium chloride, citric acid, ascorbic acid, benzoic acid, sodium carbonate, potassium carbonate, sodium bicarbonate, calcium carbonate, zinc carbonate, sodium hydroxide, potassium hydroxide, zinc hydroxide, protamine sulfate, spermine, choline, ethanolamine, triethanolamine, diethanolamine, Tween and pluronic.

Unless otherwise defined, all technical and scientific terms used herein have the same meaning as commonly understood by one of ordinary skill in the art to which this invention relates. Methods and materials are described herein for use in the present invention; other, suitable methods and materials known in the art can also be used. The materials, methods, and examples are only illustrative of the invention and, therefore, they are not intended to be limiting. All publications, patent applications, patents, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE FIGURES

FIG. 1 depicts the general structure of (3R,5R) 3,5-dihydroxyhexanoic acid oligomers. Selected HMBC correlations are shown.

FIG. 2 depicts the negative-ion ESI mass spectra of two fractions from a size-exclusion column of the oligomers.

FIG. 3 depicts the negative-ion ESIMS total ion chromatogram of Sephadex fraction 15 containing a mixture of oligomers (top), and chromatograms of selected ions corresponding to individual oligomers.

### DETAILED DESCRIPTION OF THE INVENTION

Definitions

The term “aliphatic” is an art-recognized term and includes linear, branched, and cyclic alkanes, alkenes, or alkynes. In certain embodiments, aliphatic groups in the present invention are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure. The term “alkyl” is also defined to include halosubstituted alkyls.

The term “aralkyl” is art-recognized, and includes alkyl groups substituted with an aryl group (e.g., an aromatic or heteroaromatic group).

The terms “alkenyl” and “alkynyl” are art-recognized, and include unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to ten carbons, alternatively from one to about six carbon atoms in its backbone structure. Likewise, “lower alkenyl” and “lower alkynyl” have similar chain lengths.

The term “heteroatom” is art-recognized, and includes an atom of any element other than carbon or hydrogen. Illustrative heteroatoms include boron, nitrogen, oxygen, phosphorus, sulfur and selenium, and alternatively oxygen, nitrogen or sulfur.

The term “aryl” is art-recognized, and includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxy, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and apply to 1,2-, 1,3- and 1,4-disubstituted benzenes, respectively. For example, the names 1,2-dimethylbenzene and ortho-dimethylbenzene are synonymous.

The terms “heterocyclyl” and “heterocyclic group” are art-recognized, and include 3- to about 10-membered ring structures, such as 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” and “polycyclic group” are art-recognized, and include structures with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms, e.g., three or more atoms are common to both rings, are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art recognized and includes an aromatic or non-aromatic ring in which each atom of the ring is carbon. The flowing art-recognized terms have the following meanings: “nitro” means —NO₂; the term “halogen” designates —F, —Cl, —Br or —I; the term “sulfhydryl” means —SH; the term “hydroxyl” means —OH; and the term “sulfonyl” means —SO₂⁻.

The terms “amine” and “amino” are art-recognized and include both unsubstituted and substituted amines, e.g., a moiety that may be represented by the general formulas:

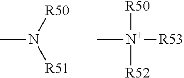

wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and includes a moiety that may be represented by the general formula:

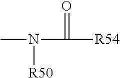

wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula:

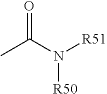

wherein R50 and R51 are as defined above. Certain embodiments of the amide in the present invention will not include imides which may be unstable.

The term “alkylthio” is art recognized and includes an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carbonyl” is art recognized and includes such moieties as may be represented by the general formulas:

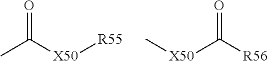

wherein X50 is a bond or represents an oxygen or a sulfur, and R55 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiocarbonyl” group. Where X50 is a sulfur and R55 or R56 is not hydrogen, the formula represents a “thioester.” Where X50 is a sulfur and R55 is hydrogen, the formula represents a “thiocarboxylic acid.” Where X50 is a sulfur and R56 is hydrogen, the formula represents a “thioformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art recognized and include an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and includes a moiety that may be represented by the general formula:

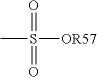

in which R57 is an electron pair, hydrogen, alkyl, cycloalkyl, or aryl.

The term “sulfate” is art recognized and includes a moiety that may be represented by the general formula:

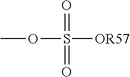

in which R57 is as defined above.

The term “sulfonamido” is art recognized and includes a moiety that may be represented by the general formula:

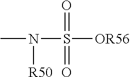

in which R50 and R56 are as defined above.

The term “sulfamoyl” is art-recognized and includes a moiety that may be represented by the general formula:

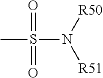

in which R50 and R51 are as defined above.

The term “sulfonyl” is art recognized and includes a moiety that may be represented by the general formula:

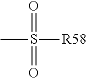

in which R58 is one of the following: hydrogen, alkyl, alkenyl, alkynyl, cycloalkyl, heterocyclyl, aryl or heteroaryl.

The term “sulfoxido” is art recognized and includes a moiety that may be represented by the general formula:

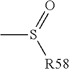

in which R58 is defined above.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, etc., when it occurs more than once in any structure, is intended to be independent of its definition elsewhere in the same structure unless otherwise indicated expressly or by the context.

Certain monomeric subunits of the present invention may exist in particular geometric or stereoisomeric forms. In addition, polymers and other compositions of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The term “hydrocarbon” is art recognized and includes all permissible compounds having at least one hydrogen and one carbon atom. For example, permissible hydrocarbons include acyclic and cyclic, branched and unbranched, carbocyclic and heterocyclic, aromatic and nonaromatic organic compounds that may be substituted or unsubstituted.

The phrase “protecting group” is art recognized and includes temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed. Greene et al., Protective Groups in Organic Synthesis 2^{nd} ed., Wiley, New York, (1991).

The phrase “hydroxyl-protecting group” is art recognized and includes those groups intended to protect a hydroxyl group against undesirable reactions during synthetic procedures and includes, for example, benzyl or other suitable esters or ethers groups known in the art.

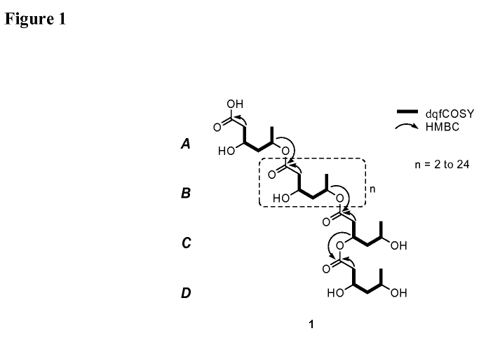

One aspect of the invention relates to a polymer excreted by an endophytic fungus from Costa. Rica. The fungus excretes large amounts of poly((3R,5R)-3,5-dihydroxyhexanoic acid) (poly(3,5-DHH)) (FIG. 1). This polymer, which contains a monomer not previously reported as a component of PHAs, shows an interesting pattern of connectivity at the hydroxy end which suggests an interesting biosynthetic route.

As part of a recent study, endophytic fungi were isolated from tissue samples of 65 plants collected in the Guanacaste Conservation Area of Costa Rica, a country containing some 4% of the world's biodiversity, including an estimated 64,000 undescribed fungi.⁶ Extracts of the 686 fungal isolates obtained were screened against a number of important drug targets. The lysate of one fungus, CR873, isolated from Manilkara sp., showed strong inhibition of IκB kinase (IKK)—a vital kinase in the NFκB signaling pathway. While attempting to identify the source of the activity against IKK, we observed a number of dihydroxyhexanoic acid derivatives which together comprised a significant fraction of the fungal extract.

CR873 was identified by rDNA sequence as having 99% identity to the ascomycete Daldinia concentrica. A liquid culture of CR873 grown in potato dextrose broth was subjected to solid-phase extraction using Diaion HP-20 resin (yield 0.269 g extract/L culture). This organic extract was then subjected to size-exclusion chromatography on Sephadex LH-20 (methanol eluent). Poly(3,5-dihydroxyhexanoic acid) (1) was obtained as the first twenty fractions from the column, accounting for 29% of the total organic extract.

1H NMR and 1H-IH double quantum filtered COSY (dqfCOSY) spectra of 1 revealed four related units, A, B, C, and D with a common spin system, CH3C(X)HCH2C(Y)HCH2-. Corresponding 13C shifts were determined by HMQC, and both IH and 13C chemical shift data were consistent with X and Y being oxygen atoms. HMBC cross-peaks from methylene protons H-2 (S 2.3 9, 2.48 in A; 2.42, 2.51 in B; 2.62, 2.69 in C and 2.42, 2.50 in D) to their respective carbon C-1(S 175.7 in A, 172.4 in B, 171.5 in C, and 172.7 in D) indicated the presence of a carbonyl adjacent to the C-2 methylene in each system. An HMBC cross-peak between H-5 (S 5.10) and C-1 (S 172.4) of B suggested an ester linkage from one B unit to another B unit. An HMBC cross-peak from H-3 of C (S 5.38) to C-1 of D (S 172.7) suggested an ester linkage connecting C to D. Neither of the methine protons of D showed HM 3C crosspeaks to suggest another ester linkage, and the chemical shift values for H-3 (4.16) and H-5 (S 3.94) are consistent with free hydroxyls at those positions in D. Chemical shifts of A were virtually identical to those of B, with the exception of the 13C chemical shift of carbon C-1 (S 175.7), which is consistent with a free acid at that position.

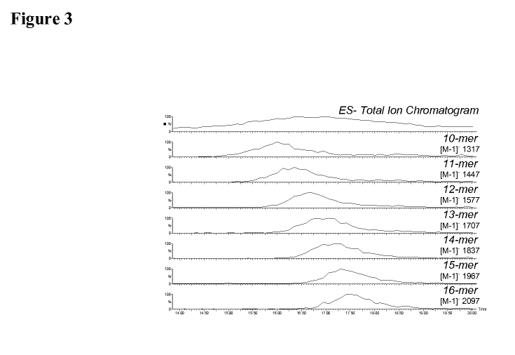

IH NMR peak integration indicated that B is by far the major component, and that A, C and D units are present in only minor amounts. The precise ratio between major B unit and minor A, C, and D units varies between the different fractions from the Sephadex column, with earlier fractions containing a larger proportion of B. Taken together, these observations suggest that the molecules are oligomers of dihydroxyhexanoic acid. A is the carboxylic acid terminus, while B appears to be the main repeating unit. D, which has no further ester bonds, appears to be the diol terminus. Interestingly, the 3-OH-linked C unit is always observed between a 5-OH-linked B unit and the terminal D unit (giving the (1,3) terminal linkage shown in FIG. 3.3). One possible explanation is that the biosynthetic machinery utilizes a 3-OH-linked dihydroxyhexanoic acid dieter as a handle for priming poly(3,5-DHH) biosynthesis with extension occurring by way of 5-OH-linked esterifications at the carboxylic acid (A) end. of the growing polymer. This variation in esterification site has not been reported in other PHAs. However, neither poly(3-HB) nor any of the other well-documented PHAs have structures that afford the option of a distinct starter unit.

Negative ion mass spectra of the polymer-containing Sephadex fractions revealed a large number of molecular ions (M−1)″, which adhered to the formula M=(130×m)+18, where m=5, b, 7, . . . , 27. These masses are consistent with linear oligomers of dihydroxyhexanoic acid ranging in size from a pentamer up to a 27-mer. As expected, higher molecular weight oligomers eluted earlier from the Sephadex column. For example, the largest peak in the mass spectrum of Fraction 10 is at (M−1) 2357, corresponding to a linear 18-mer, while the largest peak in the mass spectrum of Fraction 15 is at (M−1)−927, corresponding to a 7-mer (FIG. 3.4).

In order to confirm that the oligomers were not simply artifacts generated by the mass spectrometer, LC-MS of several of the Sephadex fractions was performed. While the separation of the oligomers on C18 was not sufficient to show individual peaks in the total ion chromatogram, comparison of the traces of different ions revealed sequential elution of individual oligomers, with longer oligomers showing a slightly longer retention time on the C18 column (FIG. 3.5).

Hydrolysis of poly(3,5-DHH) under basic conditions gave the monomer 3,5-dihydroxyhexanoic acid (2), which cyclized to form the δ-lactone (3) upon acidification and subsequent lyophilization. Lactone 3 was purified by silica gel chromatography (5:1 ethyl acetate/hexanes eluent). Analysis of the coupling constants observed in the 1H NMR spectrum of 3 in CDCl3 revealed the anti-relationship shown in FIG. 3.6. The strong coupling (J=11.3 Hz) between H4ax (δ 1.74) and H-5 (δ 4.85) indicated that both these protons are in pseudoaxia positions, while the weaker coupling (J=3.3 Hz) between H4ax (δ 1.74) and H-3 (δ 4.39) indicates a pseudoequatorial H-3. A comparison of the optical rotation of 3 ([a]25D+26.2 (c 0.2, CHC13)) with literature values* confirmed the absolute stereochemistry shown in FIGS. 3.6. 13,14

While not being bound by any particular theory, it is believed that poly(3,5-DHH) fulfills a role similar to that suggested for poly(3-HB) in bacteria: a temporary storage of reduced carbon in a form that can be easily oxidized later. Both poly(3,5-DHH) and poly(3-HB) come from acetyl-CoA that is intercepted before it enters the citric acid cycle. However, while poly(3-HB) is typically sequestered inside the bacterial cell as insoluble granules, poly(3,5-DHH) is excreted from the cell as relatively low molecular weight and water soluble polymers. The fungus feeds on this extracellular carbon source by excreting enzymes on an as needed basis. This phenomenon could also reflect a resource competition strategy whereby some organisms utilize fermentation (high consumption rate, low ATP production) to out-compete those organisms using respiration (low consumption rate, high ATP production) for resources. 11-17 Since the end product of fermentation is stored in a form inaccessible to the members using respiration, in this case a polyester, the fermenters can eliminate respirators in the short run and return to the storage metabolite in the long run.

Thus, dihydroxyhexanoic acid-derived oligomers are produced in large quantities by the ascomycete CR873. The large-scale production of biopolyesters by a eukaryote has not previously been reported. Since this initial study looked only at molecules excreted into the medium, only oligomers that were small enough to escape the fungal cell wall could be observed, leaving the possibility that larger polymers are produced and remain inside the cell.

The present invention relates to a novel polymer, a portion of which is represented by Formula 1:

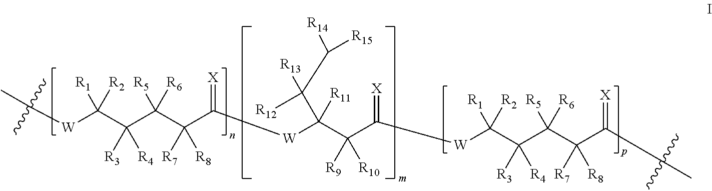

or a corresponding salt thereof, wherein:

X is independently for each occurrence O or S;

W is independently for each occurrence O, S, or NR₁₈;

R₁, R₅, and R₁₄ are each independently selected from the group consisting of OR₁₆, —O(O)R₁₆—CO(O)R₁₆, —C(O)NR₁₆R₁₇; —NR₁₆R₁₇, —NR(C)(O)R₁₆, sulfonamido, sulfoxy, sulfamyl, and halo;

R₂, R₃, R₄, R₆, R₇, R₈, R₉, R₁₀, R₁₁, R₁₂, R₁₃, and R₁₅ are each independently for each occurrence H, alkyl, alkyenyl, alkynyl, cycloalkyl, aryl, heterocyclyl, and heteroaryl;

R₁₆, R₁₇ and R₁₈ are each independently selected from the group consisting of alkyl, alkyenyl, alkynyl, cycloalkyl, aryl, heterocyclyl, and heteroaryl;

n, m and p are each independently an integer from 0 to 500, provided that n+m+p is equal to at least 2; and

wherein any alkyl, alkyenyl, alkynyl, cycloalkyl, aryl, heterocyclyl, or heteroaryl may optionally be substituted with hydroxy, alkoxy, alkenyloxy, alkynyloxy, aryloxy, aralkyloxy; halide, formyl, acetyl, halo, carboxyl, ester, cyano, nitro, SH, amino, amido, sulfonyl, or sulfonamido.

In another embodiment, the polymer represented by Formula II:

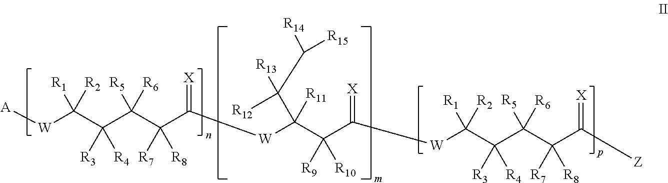

or a corresponding salt thereof,

wherein:

X is independently for each occurrence O or S;

W is independently for each occurrence O, S, or NR₁₈;

R₁, R₅, and R₁₄ are each independently selected from the group consisting of OR₁₆, —O(O)R₁₆—CO(O)R₁₆, —C(O)NR₁₆R₁₇; —NR₁₆R₁₇, —NR(C)(O)R₁₆, sulfoamido, sulfoxy, sulfamyl, nitro, cyano, and halo;

R₂, R₃, R₄, R₆, R₇, R₈, R₉, R₁₀, R₁₁, R₁₂, R₁₃, and R₁₅ are each independently for each occurrence H, alkyl, alkyenyl, alkynyl, cycloalkyl, aryl, heterocyclyl, and heteroaryl;

R₁₆, R₁₇ and R₁₈ are each independently selected from the group consisting of H, alkyl, alkyenyl, alkynyl, cycloalkyl, aryl, heterocyclyl, and heteroaryl;

n, m and p are each independently an integer from 0 to 500, provided that n+m+p is equal to at least 2; and

A and Z are independently for each occurrence selected from the group consisting of OR₁₆, —O(O)R₁₆—CO(O)R₁₆, —C(O)NR₁₆R₁₇; —NR₁₆R₁₇, —NR(C)(O)R₁₆, sulfoamido, sulfoxy, sulfamyl, nitro, cyano, halo; H, alkyl, alkyenyl, alkynyl, cycloalkyl, aryl, heterocyclyl, and heteroaryl; and

wherein any alkyl, alkyenyl, alkynyl, cycloalkyl, aryl, heterocyclyl, or heteroaryl may optionally be substituted with hydroxy, alkoxy, alkenyloxy, alkynyloxy, aryloxy, aralkyloxy; halide, formyl, acetyl, halo, carboxyl, ester, cyano, nitro, SH, amino, amido, sulfonyl, or sulfonamido.

In some embodiment, the polymers are represented by formulas I and II, wherein X and W are each O.

In some embodiments, R₁, R₅, and R₁₄ are for each occurrence OR₁₆.

In some embodiments, R₁, R₅, and R₁₄ are for each occurrence OH.

In some embodiments, R₂, R₃, R₄, R₆, R₇, R₈, R₉, R₁₀, R₁₁, R₁₂, and R₁₃ are for each occurrence H.

In some embodiments, n+m+p is equal to 5 to 250.

In some embodiments, n+m+p is equal to 5 to 100.

In some embodiments, n+m+p is equal to 5 to 50.

In some embodiments, n+m+p is equal to 5 to 25.

In some embodiments, m is equal to at least 1.

The polymers of the present invention are biodegradable. As used herein, the term “biodegradable” refers to materials that are capable of being broken down, especially into innocuous products. In some instances, the biodegradable polymers are broken down by the action of living things, such as microorganisms. The biodegradable polymers can be degraded aerobically, with oxygen, or anaerobically, without oxygen.

The aforementioned polymers have bioplastic properties, and are suitable for use in a variety of articles of manufacture. In particular, the polymers may be used in medical devices, consumer packaging, disposable diapers, garbage bags and food packaging.

In some embodiments, the polymer is soluble is a suitable solvent, such as alcohols (e.g. methanol or ethanol). Such soluble polymers can be processed to form an article of manufacture without using injection molding techniques.

In other embodiments, the polymer is a thermoplastic, and may processed to form an article of manufacture using injection molding techniques.

In some embodiments, the polymer of the present invention has a molecular weight of about 200-100,000, 200 to 50,000, 500 to 25,000, 500 to 10,000 or 700 to 3000.

In some embodiments, R₁, R₅, and R₁₄ are independently for each occurrence selected from the group consisting of O(O)R₁₆—CO(O)R₁₆, —NR₁₆R₁₇, —NR(C)(O)R₁₆, sulfonamido, sulfoxy, and sulfamyl.

In some embodiments, A is H or alkyl, and Z are OR₁₆.

In another aspect of the invention, the polymer is represented by Formula IV:

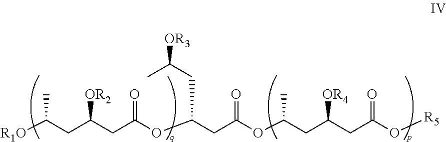

or a corresponding salt thereof,

wherein:

R₁, R₂, R₃, R₄, and R₅ are independently for each occurrence selected from the group consisting of H, alkyl, alkyenyl, alkynyl, cycloalkyl, aryl, heterocyclyl, and heteroaryl;

wherein any alkyl, alkyenyl, alkynyl, cycloalkyl, aryl, heterocyclyl, or heteroaryl may optionally be substituted with hydroxy, alkoxy, alkenyloxy, alkynyloxy, aryloxy, aralkyloxy; halide, formyl, acetyl, halo, carboxyl, ester, cyano, nitro, SH, amino, amido, sulfonyl, or sulfonamido; and

wherein p and q are each independently an integer from 1 to 500.

In some embodiments, p is equal to 1, and q is an integer from 1 to 500.

In some embodiments, q is an integer from 5 to 25.

In some embodiments, R₁, R₂, R₃, R₄, and R₅ are H.

In another aspect of the invention, the polymer comprises polymerized monomers represented by formula V:

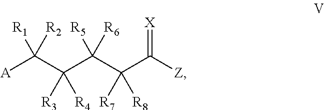

or a corresponding salt thereof,

wherein:

X is independently for each occurrence O or S;

W is independently for each occurrence O, S, or NR₁₈;

R₁, R₅, and R₁₄ are each independently selected from the group consisting of OR₁₆, —O(O)R₁₆—CO(O)R₁₆, —C(O)NR₁₆R₁₇; —NR₁₆R₁₇, —NR(C)(O)R₁₆, sulfonamido, sulfoxy, sulfamyl, nitro, cyano, and halo;

R₂, R₃, R₄, R₆, R₇, R₈, R₉, R₁₀, R₁₁, R₁₂, R₁₃, and R₁₅ are each independently for each occurrence H, alkyl, alkyenyl, alkynyl, cycloalkyl, aryl, heterocyclyl, and heteroaryl;

R₁₆, R₁₇ and R₁₈ are each independently selected from the group consisting of H, alkyl, alkyenyl, alkynyl, cycloalkyl, aryl, heterocyclyl, and heteroaryl;

n, m and p are each independently an integer from 0 to 500, provided that n+m+p is equal to at least 2; and

A and Z are independently for each occurrence selected from the group consisting of OR₁₆, SR₁₆, —O(O)R₁₆; —NHR₁₆, —NR(C)(O)R₁₆, and halo; and

wherein any alkyl, alkyenyl, alkynyl, cycloalkyl, aryl, heterocyclyl, or heteroaryl may optionally be substituted with hydroxy, alkoxy, alkenyloxy, alkynyloxy, aryloxy, aralkyloxy; halide, formyl, acetyl, halo, carboxyl, ester, cyano, nitro, SH, amino, amido, sulfonyl, or sulfonamido.

In some embodiments, the monomers are attached via an ester, thioester, or amide linkage.

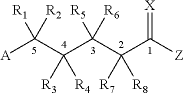

In some embodiments, at least one monomer is attached via an ester, thioester, or amide linkage at the 3 position, and the remaining monomers are connected at the 5 position, as shown above.

In some embodiments, the polymer has a molecular weight of about 200 to 200,000, 200 to 50,000, 200 to 25,000, 200 to 10,000, or 200 to 5,000.

In some embodiments, the monomer represented by formula V is copolymerized with at least one additional monomer selected from the group consisting of hydroxy acids, amino acids, aminoalcohols, sugars, diols, and triols, tetraols.

In some embodiments, the additional monomer is selected from the group consisting of glycolic acid, lactic acid, 2-hydroxyethoxy acetic acid, polyetheylene glycol and hyarulonic acid.

Another aspect if the invention is directed to a method of making a polymer comprising extracting a fungus to produce the polymer.

In some embodiments, the fungus is an endophytic fungus.

In some embodiments, the fungus is CR873.

In some embodiments, the method further comprises culturing the fungus prior to extraction. For example, the CR873 may be cultured in a suitable culture medium and harvesting by extracting the polymers from culture medium with a suitable solvent, concentrating the solution containing the desired component, and subjecting the concentrated material to chromatographic separation to isolate the desired polymers from other metabolites also present in the cultivation medium. Broadly, the culture medium includes glucose, fructose, mannose, maltose, galactose, mannitol and glycerol, other sugars and sugar alcohols, starches and other carbohydrates, or carbohydrate derivatives, such as dextran, cerelose, as well as complex nutrients, such as oat flour, com meal, millet, corn and the like. The exact quantity of the carbon source which is utilized in the medium will depend, in part, upon the other ingredients in the medium, but it is usually found that an amount of carbohydrate between about 0.5 and 15 percent by weight of the medium is satisfactory. These carbon sources can be used individually or several such carbon sources may be combined in the same medium. In some embodiments, the culture medium includes potato starch.

In some embodiments, the fungus is capable of performing a biosynthetic pathway to produce any of the aforementioned polymers.

In some embodiments, the extraction is a solid phase extraction.

In some embodiments, the polymer comprises at least one hydroxy group, and the method further comprises chemically modifying the hydroxy group.

In some embodiments, the hydroxy group is modified to a group selected from OR₁₆, —O(O)R₁₆—CO(O)R₁₆, —C(O)NR₁₆R₁₇; —NR₁₆R₁₇, —NR(C)(O)R₁₆, sulfonamido, sulfoxy, sulfamyl, nitro, cyano, and halo,

wherein R₁₆, and R₁₇ are each independently selected from the group consisting of H, alkyl, alkyenyl, alkynyl, cycloalkyl, aryl, heterocyclyl, and heteroaryl; and

wherein any alkyl, alkyenyl, alkynyl, cycloalkyl, aryl, heterocyclyl, or heteroaryl may optionally be substituted with hydroxy, alkoxy, alkenyloxy, alkynyloxy, aryloxy, aralkyloxy; halide, formyl, acetyl, halo, carboxyl, ester, cyano, nitro, SH, amino, amido, sulfonyl, or sulfonamido.

Another aspect of the invention relates to a polymer, which is the product produced by any of the aforementioned processes. Another aspect of the invention relates to a cultural filtrate of a fungus, such as an endophytic fungus. In some embodiments, the fungus is CR873. In certain embodiments, it may be preferential to utilize polymer compounds which have not been purified from the culture filtrate in which they were formed. In other words, one aspect of the invention is the formation and use of a cultural filtrate of a CR873 culture.

The polymers of the present invention can be formulated into bioplastic comprising the polymer and at least one additive. The additives can alter the properties of the bioplastic composition as needed for use in certain articles of manufacture. For example, implantable medical devices can be formulated to biodegrade with in several hours, several days, several weeks, several months, or several years. Similarly, consumer packaging or product bioplastic compositions can be formulate to biodegrade in landfills within several days, several weeks, several months, or several years.

Accordingly, another aspect of the invention relates to a bioplastic composition comprising a polymer of the present invention.

In some embodiments, the bioplastic composition further comprises at least one additive.

In some embodiments, the additive increases the biodegradability of the polymer.

In some embodiments, the additive decreases the biodegradability of the polymer.

In some embodiments, the additive is selected from the group consisting of inorganic acids, inorganic bases, ammonium salts, organic acids, and surfactants.

In some embodiments, the additive is selected from the group consisting ammonium sulfate, ammonium chloride, citric acid, ascorbic acid, benzoic acid, sodium carbonate, potassium carbonate, sodium bicarbonate, calcium carbonate, zinc carbonate, sodium hydroxide, potassium hydroxide, zinc hydroxide, protamine sulfate, spermine, choline, ethanolamine, triethanolamine, diethanolamine, Tween and pluronic.

### Examples

General Experimental Procedures: NMR spectra of the oligomers (1) were measured using a Varian NOVA 600 MHz spectrometer equipped with a 5 mm triple gradient HCN probe. All other NMR spectra were measured using a Varian System 600 MHz spectrometer. 1 H and 13C chemical shifts of 1 were referenced with the methanol solvent peaks at δ 3.31 and δ 49.0, respectively; chemical shifts of 3 were referenced with the chloroform solvent peaks at δ 7.26 and δ 77.0. IR spectra were recorded on a Mattson Galaxy Series 3000 FTIR spectrometer. Optical rotation data were obtained using a Jasco DIP-370 polarimeter. Low resolution mass spectra were performed on a Micromass Quattro spectrometer operating in negative ion electrospray mode. The high resolution mass spectrum of 3 was run by the University of Illinois Mass Spectrometry facility. Adsorption chromatography was carried out on silica gel 60 (FisherChemicals, 230-400 mesh). Size exclusion chromatography was carried out on Sephadex″u LH-20 (Amersham Biosciences).

Fungal material: CR873 was isolated from the interior of a Manilkara sp. leaf harvested from the Guanacaste Conservation Area in Costa Rica. (Collection notesindicate that the leaf was uneaten, but brown around the edges.) Plant tissue was surface sterilized by successive 5-minute washes in 10% bleach, 70% ethanol, and three fresh portions of sterile water. Five-mm squares of plant tissue were cut from the surface-sterilized specimen and placed on PDA plates to promote fungal growth. CR873 was subcultured from the fungi that grew from these samples after two days and then successively subcultured to obtain a pure fungal culture.

CR873 rDNA Sequencing. Genomic DNA was extracted from lyophilized fungal tissue by grinding in liquid nitrogen followed by hot lysis (65° C., 0.5% SDS), phenol/chloroform extraction, and ethanol precipitation. Thus obtained, CR873 genomic DNA was used as the template for a polymerase chain reaction with primers LROR (5′-ACCCGCTGAACTTAAGC-3′) and LR5 (5′-TCCTGAGGGAAACTTCG3′) to amplify a 900 base pair fragment of the large subunit ribosomal DNA. The PCR product was cloned and sequenced, and the consensus sequence used in a BLASTsearch to identify closely related fungi.

Culture Conditions: Agar plugs of CR873 on potato dextrose agar were used to inoculate seed cultures in potato dextrose broth at 25° C. The four-day old seed cultures were then used to inoculate 10 L fermenter flasks containing potato. Dextrose broth. After fermenting for 7 days at 25° C., the fungal cultures were temporarily stored at −20° C. prior to extraction.

Extraction and isolation: Solid-phase extraction of a 20 L culture of CR873 grown in potato dextrose broth gave 5.39 g total organic extract. Size-exclusion chromatography on Sephadex LH-20 gave the poly(3,5-DHH) (1) as the first twenty fractions from the column (1.58 g total).

3,5-dihydroxyhezanoic acid S-lactone (3) was obtained as an optically active clear glass, [a]25D±26.2 (c 0.2, CHC13); IR (NaCl, thin film) vm.: 1708, 1387, 1258, 1073 cm 1; 13C NUR (CDC13, 600 MHz, 8): 170.2 (C-1), 72.1(C-5), 62.9 (C-3), 38.4 (C-4), 37.7 (C-2), 21.3 (C-6);111 NMR (CDC13, 600-MHz, 8): 1.40 (d, J6-5=6.5 Hz, 3H, H6), 1.74 (ddd, J4, . . . 4 . . . 4=14.5 Hz, J4ax-5=11.3 Hz, J4 . . . 3=3.3 Hz, 1H, H-4ax), 1.97 (dddd, J4eq.4.=14.5 Hz, J4eq_{—}3=3.8 Hz, J4eq_{—}5=3.0 Hz, J4eq_{—}2eq=1.8 Hz, 111, H-4eq), 2.61(ddd, J2e9_{—}2ax=17.6 Hz, J2,q_{—}3=3.7 Hz, J2eq.4ey=1.8 Hz, 111, H-2eq), 2.74 (dd, J2=_{—}2eq=17.6 Hz, J2.,-3=5.1 Hz, 111, H-2ax), 4.39 (dqd, J3_{—}2 . . . =5.1 Hz, J3-2.q=J34a.

J3-4.q=3.8 Hz, J3_{—}5=0.5 Hz, 111, H-3),4.85 (dgdd, J5-4.=11.2 Hz, J5_{—}6=6.5 Hz, J5. 4N=3.0 Hz, JS_{—}3=0.5 Hz, 1H, H-5). HRMS-EI+ (mlz): [M+1]+ calcd for C6HI003, 130.0630; found, 130.0631.

¹H NMR and ¹H-¹H double quantum filtered COSY (dqfCOSY) spectra revealed four related units, A, B, C, and D with a common spin system, CH₃CHCH₂CHCH₂—. Corresponding ¹³C shifts were determined by HMQC, and both ¹H and ¹³C chemical shift data were consistent with oxygen atoms at carbons C-3 and C-5 of each unit. HMBC cross-peaks from methylene protons H-2 (δ 2.39, 2.48 in A; 2.42, 2.51 in B; 2.62, 2.69 in C; and 2.42, 2.50 in D) to their respective carbon C-1 (δ 175.7 in A, 172.4 in B, 171.5 in C, and 172.7 in D) indicated the presence of a carbonyl adjacent to the C-2 methylene in each system. An HMBC cross-peak between H-5 (δ 5.10) and C-1 (δ 172.4) of B suggested an ester linkage from one B unit to another B unit. An HMBC cross-peak from H-3 of C (δ 5.38) to C-1 of D (δ 172.7) suggested an ester linkage connecting C to D. Neither of the methine protons of D showed HMBC crosspeaks to suggest another ester linkage, and the chemical shift values for H-3 (δ 4.16) and H-5 (δ 3.94) are consistent with free hydroxyls at those positions in D. Chemical shifts of A were virtually identical to those of B, with the exception of the ¹³C chemical shift of carbon C-1 (δ 175.7), which is consistent with a free acid at that position.

¹H NMR peak integration indicates that B is by far the major component, and that A, C and D units are present in only minor amounts. The precise ratio between major B unit and minor A, C, and D units varies between the different fractions from the Sephadex column, with earlier fractions containing a larger proportion of B. Taken together, these observations suggest that the molecules are oligomers of dihydroxyhexanoic acid. A is the carboxylic acid terminus, while B appears to be the main repeating unit. D, which has no further ester bonds, appears to be the alcohol terminus. Interestingly, the 3-OH-linked C unit is always observed between a 5-OH-linked B unit and the terminal D unit (FIG. 1). This variation in esterification site has not been observed in other PHAs. One possible explanation is that the oligomer biosynthetic machinery utilizes a 3-OH-linked dihydroxyhexanoic acid dimer as a handle for priming oligomer biosynthesis, with extension occurring by way of 5-OH-linked esterifications at the carboxylic acid (A) end of the growing oligomer. Alternatively, if the oligomers are biosynthesized by a mechanism involving extension from the alcohol end, accidental addition to the 3-hydroxy position could prevent further chain extension.

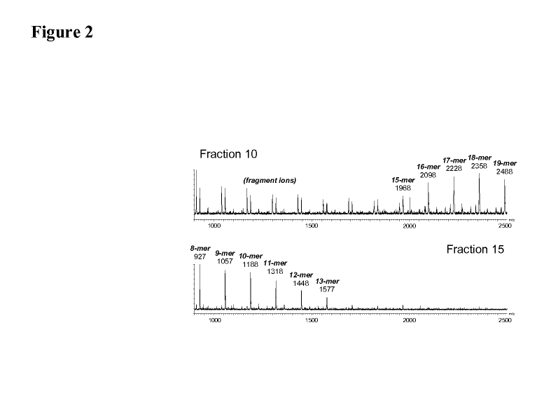

Negative ion mass spectra of the oligomer-containing Sephadex fractions revealed a large number of molecular ions (M−1)⁻, which adhered to the formula M=(130·x)+18, where x=5, 6, 7, . . . , 27. These masses are consistent with linear oligomers of dihydroxyhexanoic acid ranging in size from a pentamer up to a 27-mer. As expected, higher molecular weight oligomers eluted earlier from the Sephadex column. For example, the largest peak in the mass spectrum of Fraction 10 is at (M−1)⁻ 2357, corresponding to a linear 18-mer, while the largest peak in the mass spectrum of Fraction 15 is at (M−1)⁻ 927, corresponding to an 8-mer (FIG. 2).

In order to confirm that the oligomers were not simply artifacts generated by the mass spectrometer, LC-MS of several of the Sephadex fractions was performed. While the separation of the oligomers on C18 was not sufficient to show individual peaks in the total ion chromatogram, comparison of the traces of different ions revealed sequential elution of individual oligomers, with longer oligomers showing a slightly longer retention time on the C18 column (FIG. 3).

Hydrolysis of the oligomers under basic conditions gave the monomer 3,5-dihydroxyhexanoic acid (2), which cyclized to form the δ-lactone (3) upon acidification and subsequent lyophilization. Lactone 3 was purified by silica gel chromatography (5:1 ethyl acetate:hexanes eluent). Analysis of the coupling constants observed in the 1H NMR spectrum of 3 in CDCl₃ revealed the anti-relationship shown in Scheme 1. The strong coupling (J=11.3 Hz) between H-4 (δ 1.74) and H-5 (4.85) indicated that both these protons are in pseudoaxial positions, while the weaker coupling (J=3.3 Hz) between H-4ax (δ 1.74) and H-3 (4.39) indicates a pseudoequatorial H-3. A comparison of the optical rotation of 3 ([α]²⁵_{D}+26.2 (c 0.2, CHCl₃)) with literature values⁷ confirmed the absolute stereochemistry shown in Scheme 1.

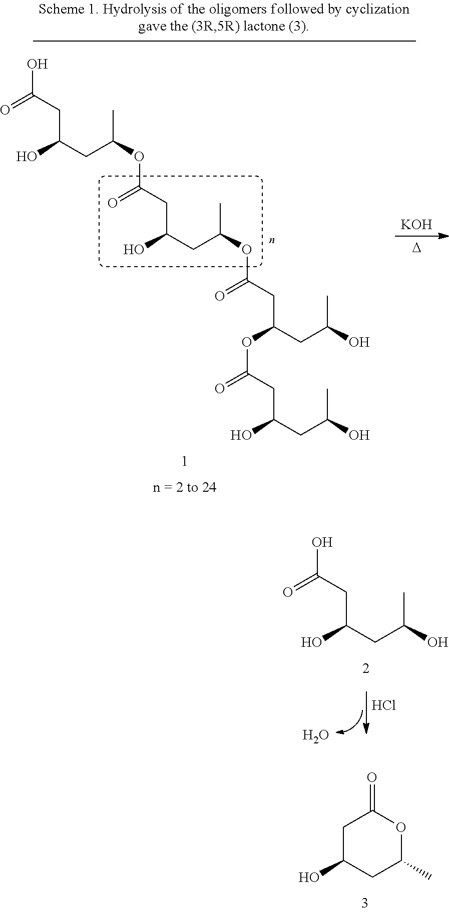

### INCORPORATION BY REFERENCE

The contents of all cited references (including literature references, issued patents, published patent applications as cited throughout this application) are hereby expressly incorporated by reference. When definitions of terms in documents that are incorporated by reference herein conflict with those used herein, the definitions used herein govern.

Equivalents

Those skilled in the art will recognize, or be able to ascertain using no more than routine experimentation, many equivalents to the specific embodiments of the invention described herein. Such equivalents are intended to be encompassed by the following claims.

### REFERENCES

1. Stubbe, J.; Tian, J.; He, A.; Sinskey, A. J.; Lawrence, A. G.; Liu, P., Nontemplate-dependent polymerization processes: polyhydroxyalkanoate synthases as a paradigm. Rev. Biochem. 2005, 74, 433-480.

2. Pool, R, In search of the plastic potato. Science 1989, 245,1187-1189.

3. Lee, S. Y., Bacterial polyhydroxyalkanoates. Biotechnol. Bioeng. 1996, 49, (1),114.

4. Byrom, D., Polymer synthesis by microorganisms: technology and economics. Trends Biotechnol. 1987, 5, (9), 246-250.

5. Steinbiichel, A., Polyhydroxyalkanoic acids. In Biomaterials: Novel materials from biological sources, Byrom, D., Ed. Stockton Press: New York, 1991, 125-213.

6. Madison, L. L.; Huisman, G. W., Metabolic engineering of poly(3-hydroxyalkanoates): from DNA to plastic. A crobiol. Mot. Biol. Rev. 1999, 63, (1), 21-53.

7. Rehm, B. H., Polyester synthases: natural catalysts for plastics. Biochem. J. 2003, 376,15-33.

8. Steinbiichel, A.; Valentin, H. E., Diversity of bacterial polyhydroxyalkanoic acids. FEMS Microbiol. Lett. 1995,128, (3), 219-228.

9. Reusch, R. N., Transmembrane ion transport by polyphosphate/poly-(R)-3-hydroxybutyrate complexes. Biochemistry (Mosc) 2000, 65, (3), 280-295.

10. Fischer, H.; Erdmann, S.; Holler, E., An unusual polyanion from Physarum polycephalum that inhibits homologous DNA polymerase alpha in vitro. Biochemistry 1989, 28, (12), 5219-5226.

11. Shimada, K.; Matsushima, K.; Fukumoto, J.; Yamamoto, T., Poly-(L)-malic acid; a new protease inhibitor from Penicillium cyclopium. Biochem. Biophys. Res. Comm. 1969, 35, (5),619-624.

12. Sittenfeld, A.; Villers, R, Exploring and preserving biodiversity in the tropics: the Costa Rican case. Curr. Opin. Biotechnol. 1993, 4, 280-285.

13. Bennett, F.; Knight, D. W.; Fenton, G., Methyl (3R)-3-hydroxyhex-5-enoate as a precursor to chiral mevinic acid analogs. J Chem. Soc. Perkin Trans. 1: Org. Bioorg. Chem. 1991,(1),133-140.

14. Le Sann, C.; Munoz, D. M.; Saunders, N.; Simpson, T. L; Smith, D. L; Soulas, F.; Watts, P.; Willis, C. L., Assembly intermediates in polyketide biosynthesis: enantioselective syntheses of beta hydroxycarbonyl compounds. Org. Biomol. Chem. 2005, 3, (9), 1719-1728.

15. Pfeiffer, T.; Schuster, S.; Bonhoeffer, S., Cooperation and competition in the evolution of ATP-producing pathways. Science 2001, 292, (5516), 504-507.

16. Kret I U., Biofilms promote altruism. Vicrobiol. 2004, 150, 2751-2760.

17. MacLean, R. C.; Gudelj, I., Resource competition and social conflict in experimental populations of yeast. Nature 2006, 441, (7092), 498-501.

1. A polymer, a portion of which comprises Formula 1: 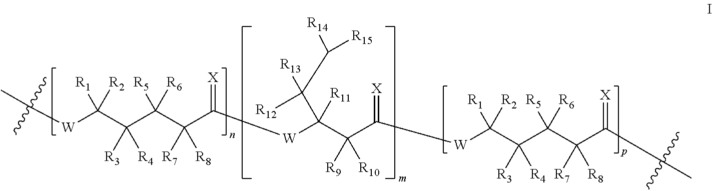
or a corresponding salt thereof, wherein:
X is independently for each occurrence O or S;
W is independently for each occurrence O, S, or NR₁₈;
R₁, R₅, and R₁₄ are each independently selected from the group consisting of OR₁₆, —O(O)R16—CO(O)R₁₆, —C(O)NR₁₆R₁₇; —NR₁₆R₁₇, —NR(C)(O)R₁₆, sulfonamido, sulfoxy, sulfamyl, and halo;
R₂, R₃, R₄, R₆, R₇, R₈, R₉, R₁₀, R₁₁, R₁₂, R₁₃, and R₁₅ are each independently for each occurrence H, alkyl, alkyenyl, alkynyl, cycloalkyl, aryl, heterocyclyl, and heteroaryl;
R₁₆, R₁₇ and R₁₈ are each independently selected from the group consisting of alkyl, alkyenyl, alkynyl, cycloalkyl, aryl, heterocyclyl, and heteroaryl;
n, m and p are each independently an integer from 0 to 500, provided that n+m+p is equal to at least 2; and
wherein any alkyl, alkyenyl, alkynyl, cycloalkyl, aryl, heterocyclyl, or heteroaryl may optionally be substituted with hydroxy, alkoxy, alkenyloxy, alkynyloxy, aryloxy, aralkyloxy;
halide, formyl, acetyl, halo, carboxyl, ester, cyano, nitro, SH, amino, amido, sulfonyl, or sulfonamido.

2. The polymer of claim 1, represented by Formula II: 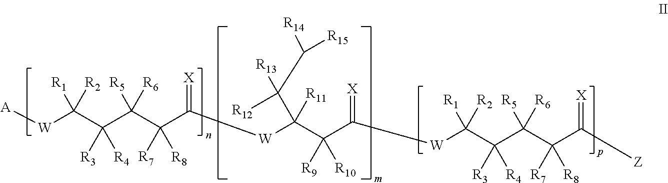
or a corresponding salt thereof,
wherein:
X is independently for each occurrence O or S;
W is independently for each occurrence O, S, or NR₁₈;
R₁, R₅, and R₁₄ are each independently selected from the group consisting of OR₁₆, —O(O)R₁₆—CO(O)R₁₆, —C(O)NR₁₆R₁₇; —NR₁₆R₁₇, —NR(C)(O)R₁₆, sulfonamido, sulfoxy, sulfamyl, nitro, cyano, and halo;
R₂, R₃, R₄, R₆, R₇, R₈, R₉, R₁₀, R₁₁, R₁₂, R₁₃, and R₁₅ are each independently for each occurrence H, alkyl, alkyenyl, alkynyl, cycloalkyl, aryl, heterocyclyl, and heteroaryl;
R₁₆, R₁₇ and R₁₈ are each independently selected from the group consisting of H, alkyl, alkyenyl, alkynyl, cycloalkyl, aryl, heterocyclyl, and heteroaryl;
n, m and p are each independently an integer from 0 to 500, provided that n+m+p is equal to at least 2; and
A and Z are independently for each occurrence selected from the group consisting of OR₁₆, —O(O)R₁₆—CO(O)R₁₆, —C(O)NR₁₆R₁₇; —NR₁₆R₁₇, —NR(C)(O)R₁₆, sulfonamido, sulfoxy, sulfamyl, nitro, cyano, halo; H, alkyl, alkyenyl, alkynyl, cycloalkyl, aryl, heterocyclyl, and heteroaryl; and
wherein any alkyl, alkyenyl, alkynyl, cycloalkyl, aryl, heterocyclyl, or heteroaryl may optionally be substituted with hydroxy, alkoxy, alkenyloxy, alkynyloxy, aryloxy, aralkyloxy; halide, formyl, acetyl, halo, carboxyl, ester, cyano, nitro, SH, amino, amido, sulfonyl, or sulfonamido.

3-5. (canceled)

6. The polymer of claim 1, wherein X and W are each O.

7. The polymer of claim 1, wherein R₁, R₅, and R₁₄ are for each occurrence OR₁₆.

8. The polymer of claim 1, wherein R₁, R₅, and R₁₄ are for each occurrence OH.

9. The polymer of claim 1, wherein R₂, R₃, R₄, R₆, R₇, R₈, R₉, R₁₀, R₁₁, R₁₂, and R₁₃ are for each occurrence H.

10. The polymer of claim 1, wherein n+m+p is equal to 5 to 250.

11-20. (canceled)

21. A polymer represented by Formula IV: 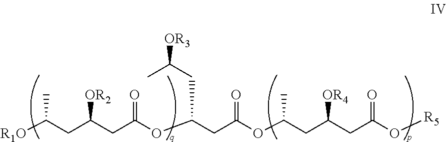
or a corresponding salt thereof,
wherein:
R₁, R₂, R₃, R₄, and R₅ are independently for each occurrence selected from the group consisting of H, alkyl, alkyenyl, alkynyl, cycloalkyl, aryl, heterocyclyl, and heteroaryl;
wherein any alkyl, alkyenyl, alkynyl, cycloalkyl, aryl, heterocyclyl, or heteroaryl may optionally be substituted with hydroxy, alkoxy, alkenyloxy, alkynyloxy, aryloxy, aralkyloxy; halide, formyl, acetyl, halo, carboxyl, ester, cyano, nitro, SH, amino, amido, sulfonyl, or sulfonamido; and
wherein p and q are each independently an integer from 1 to 500.

22. The polymer of claim 21, wherein p is equal to 1, and q is an integer from 1 to 500.

23. The polymer of claim 22, wherein q is an integer from 5 to 25.

24. The polymer of claim 21, wherein R₁, R₂, R₃, R₄, and R₅ are H.

25. A polymer comprising polymerized monomers represented by formula V: 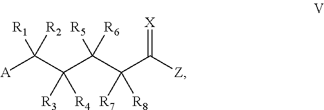
or a corresponding salt thereof,
wherein:
X is independently for each occurrence O or S;
W is independently for each occurrence O, S, or NR₁₈;
R₁, R₅, and R₁₄ are each independently selected from the group consisting of OR₁₆, —O(O)R₁₆—CO(O)R₁₆, —C(O)NR₁₆R₁₇; —NR₁₆R₁₇, —NR(C)(O)R₁₆, sulfonamido, sulfoxy, sulfamyl, nitro, cyano, and halo;
R₂, R₃, R₄, R₆, R₇, R₈, R₉, R₁₀, R₁₁, R₁₂, R₁₃, and R₁₅ are each independently for each occurrence H, alkyl, alkyenyl, alkynyl, cycloalkyl, aryl, heterocyclyl, and heteroaryl;
R₁₆, R₁₇ and R₁₈ are each independently selected from the group consisting of H, alkyl, alkyenyl, alkynyl, cycloalkyl, aryl, heterocyclyl, and heteroaryl;
n, m and p are each independently an integer from 0 to 500, provided that n+m+p is equal to at least 2; and
A and Z are independently for each occurrence selected from the group consisting of OR₁₆, SR₁₆, —O(O)R₁₆; —NHR₁₆, —NR(C)(O)R₁₆, and halo; and
wherein any alkyl, alkyenyl, alkynyl, cycloalkyl, aryl, heterocyclyl, or heteroaryl may optionally be substituted with hydroxy, alkoxy, alkenyloxy, alkynyloxy, aryloxy, aralkyloxy; halide, formyl, acetyl, halo, carboxyl, ester, cyano, nitro, SH, amino, amido, sulfonyl, or sulfonamido.

26. The polymer of claim 25, wherein the monomers are attached via an ester, thioester, or amide linkage.

27. The polymer of claim 25, wherein at least one monomer is attached via an ester, thioester, or amide linkage at the 3 position, and the remaining monomers are connected at the 5 position.

28-32. (canceled)

33. The polymer of claim 25, wherein in the monomer represented by formula V is copolymerized with at least one additional monomer selected from the group consisting of hydroxy acids, amino acids, aminoalcohols, sugars, diols, and triols, tetraols.

34. The polymer of claim 25, wherein the additional monomer is selected from the group consisting of glycolic acid, lactic acid, 2-hydroxyethoxy acetic acid, polyetheylene glycol and hyarulonic acid.

35. A method of making a polymer comprising:
extracting a fungus, wherein the fungus is CR873.

36-37. (canceled)

38. The method of claim 35, further comprising culturing the fungus prior to extraction.

39. The method of claim 38, wherein the fungus is cultured in a suitable culture medium comprising a carbon source selected from the group consisting of glucose, fructose, mannose, maltose, galactose, mannitol and glycerol, sugars, sugar alcohols, starches, carbohydrates, carbohydrate derivatives, dextran, cerelose, or potato starch.

40-41. (canceled)

42. The method of claim 39, wherein the polymer produced by the biosynthetic pathway has the following formula IV: 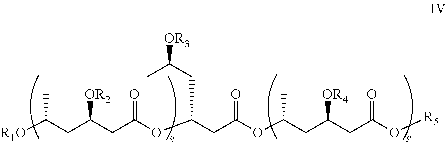
or a corresponding salt thereof,
wherein:
R₁, R₂, R₃, R₄, and R₅ are independently for each occurrence selected from the group consisting of H, alkyl, alkenyl, alkynyl, cycloalkyl, aryl, heterocyclyl, and heteroaryl;
wherein any alkyl, alkynyl, alkynyl, cycloalkyl, aryl, heterocyclyl, or heteroaryl may optionally be substituted with hydroxy, alkoxy, alkenyloxy, alkynyloxy, aryloxy, aralkyloxy; halide, formyl, acetyl, halo, carboxyl, ester, cyano, nitro, SH, amino, amido, sulfonyl, or sulfonamido; and
wherein p and q are each independently an integer from 1 to 500.

43-52. (canceled)

53. A culture comprising CR873.

54. (canceled)
